(19) United States
(12) Patent Application Publication
(10) Pub. No.: US 2025/0041586 A1
(43) Pub. Date: Feb. 6, 2025

(54) PUMP HOUSING FOR A BLOOD PUMP AND BLOOD PUMP

(71) Applicant: Abiomed Europe GmbH, Aachen (DE)

(72) Inventors: Wolfgang Kerkhoffs, Aachen (DE); Ellen Keysselitz, Aachen (DE)

(21) Appl. No.: 18/717,826

(22) PCT Filed: Dec. 8, 2022
(86) PCT No.: PCT/EP2022/085057

§ 371 Date: Jun. 7, 2024

(30) Foreign Application Priority Data
Dec. 10, 2021 (DE) 102021214168.4

(51) Int. Cl. A61M 60/816

(52) U.S. Cl. CPC A61M 60/816

(57) ABSTRACT

The present invention relates to a pump housing (10) for a blood pump (100). The pump housing (10) comprises a distal end portion (12) having a first blood flow opening (14); a proximal end portion (16); an intermediate portion (18) extending axially between the distal end portion (12) and the proximal end portion (16), the intermediate portion (18) having at least one second blood flow opening (20); and a first sensor (22) for sensing at least one parameter, in particular aortic pressure; wherein the first sensor (22) is disposed on an outer peripheral surface (24) of the intermediate portion (18), wherein the pump housing (10) comprises a second sensor (26), wherein the second sensor (26) is disposed on the distal end portion (12) for sensing at least one parameter, in particular pressure at the first blood flow opening. The present invention further relates to a blood pump comprising an according pump housing (10).

The present invention relates to a pump housing for a blood pump and to a blood pump comprising a respective pump housing. The blood pump is preferably a catheter pump or an intravascular blood pump.

### BACKGROUND

Blood pumps of different types are known, such as intravascular blood pumps that may be introduced into the heart of a patient to support the blood flow from the heart into a blood vessel e.g., an artery. Intravascular blood pumps may be introduced percutaneously during a cardiac procedure through the vascular system, such as by a catheterization procedure. A blood pump typically comprises of a pump housing, a cannula and a catheter. The cannula is attached to a cannula attachment portion provided on a distal end portion of the pump housing and the catheter is attached to a proximal end portion of the pump housing. Commonly, such intravascular blood pumps are used as left ventricular assist devices, wherein the cannula reaches through the aortic valve into the left ventricle whereby the pump housing and the catheter are located outside of the heart in the aorta. A pump element in form of an impeller is disposed within the pump housing generates a suction pressure and blood is unloaded from the left ventricle into the aorta to restore adequate systemic blood flow. Therefore, the pump housing further comprises at least one blood flow opening through which the blood can exit the blood pump into the blood vessel.

A first necessity associated generally with such blood pumps is mechanical stability. In particular, any malfunction or mechanical failure needs to be detected urgently, as this might otherwise lead to dangerous situations for the patient as directly impacting the vitality and health. This not only covers a drop in the unloading capability of the blood pump, but also the detachment of particles or entities from the blood pump itself which may cause damage to the vasculature of the patient.

A second necessity is to avert suction occurrence, which might be caused in case unloading of the left ventricle is too high. Suction events lead to arrhythmias any may further cause tissue trauma within the left ventricle. In addition, suction events lead to a reduced blood flow which by itself may cause damage with the blood pump. In some implementations of blood pumps, sufficient blood flow is required to lubricate and cool the bearings of the impeller or other parts of the blood pump. Accordingly, suction events may cause secondary damage, which by itself can again entail dangerous situations for the patient's vitality and health. Of course, this needs to be avoided.

One possibility to promptly detect malfunctions and to derive conclusions on suction events is to monitor the blood pressure around the blood pump introduced into the patient's heart. WO 2020/061399 A1 and WO 2017/214118 A1 therefore suggest to provide a sensor on an outer peripheral surface of the pump housing for monitoring the aortic pressure. A detected pressure drop may deliver insights that the mechanical stability of the blood pump is no longer guaranteed and that urgent attention of the physician is required to ensure the vitality and health of the patient.

A further possibility is to monitor the power consumption of the motor, e.g. by monitoring the electric current uptake of the motor. Peaks and drops within the power consumption of the motor may deliver further insights into the operational characteristics of the blood pump and the suction pressure of the blood pump may be derived therefrom. However, monitoring power consumption is of subordinate importance in case the electric motor is not fixedly coupled to the impeller, e.g. by a shaft or the like. Recently, non-contact coupling between the motor and the impeller has been proven to be of advantage. For instance, WO 2020/187860 A1 suggests a magnetic drive unit for rotating the impeller. With such a system, peaks and drops within the power consumption cannot be sufficiently linked to the performance of the blood pump.

Therefore, the need exists to provide a further possibility to reliably detect suction events and malfunctions of blood pumps.

### SUMMARY

According to a first aspect, a pump housing for a blood pump comprises a distal end portion having a first blood flow opening, a proximal end portion, and an intermediate portion extending axially between the distal end portion and the proximal end portion. The intermediate portion has at least one second blood flow opening. The pump housing comprises a first sensor for sensing at least one parameter, in particular blood vessel pressure and preferably aortic pressure, wherein the first sensor is disposed on an outer peripheral surface of the intermediate portion. The pump housing further comprises a second sensor, wherein the second sensor is disposed on the distal end portion for sensing at least one parameter, in particular pressure at the first blood flow opening.

The second sensor is disposed up- or downstream of the first sensor and delivers a further parameter, in particular the pressure at the first blood flow opening. Thus, it is possible to not only sense the blood vessel pressure directly, but also the pressure at the first blood flow opening is directly sensed. It is not necessary to derive the pressure at the first blood flow opening from e.g. the power consumption of the motor. In case of a suction event, the pressure drop at the first blood flow opening may directly be sensed and further measures may immediately been taken to ensure the patient's vitality. Further, this also increases safety, in particular for blood pumps having a non-fixed coupling between the motor and the impeller.

The first blood flow opening may be a blood flow inlet or a blood flow outlet. Accordingly, the at least one second blood flow opening may be a blood flow outlet or a blood flow inlet. Depending on the application of the blood pump, a blood flow is thus generated from the first blood flow opening to the second blood flow opening or from the second blood flow opening to the first blood flow opening. Preferably, the blood pump is a left ventricular support so that the first blood flow opening preferably is a blood flow inlet and the at least one second blood flow opening preferably is a blood flow outlet.

The distal end portion may comprise a thickened portion and the thickened portion may extend radially inwardly from the distal end portion. The second sensor may be disposed in the thickened portion. Preferably, the distal end portion comprises a cannula attachment portion and the thickened portion may extend radially inwardly from the cannula attachment portion. Due to the thickened portion, a sufficient material thickness for supporting the second sensor is provided, decreasing the risk of detachment of the second sensor.

The thickened portion may comprise a support recess, wherein the second sensor may be disposed within the support recess. The support recess may be configured to distinctly set the position of the second sensor. This greatly facilitates the attachment of the second sensor and further warrants a correct position of the second sensor relative to the distal end portion.

The thickened portion may taper in the axial direction from the first blood flow opening to the intermediate portion. In particular, the thickened portion may smoothly taper to allow for the least possible amount of blood flow interruption.

A support member with at least two arm portions may be disposed in the distal end portion, wherein one of the arm portions may comprise the thickened portion. The support member reduces turbulences within the blood flow and the thickened portion is directly integrated into the support member or one of the arm portions respectively.

The support member may comprise a bearing support portion being concentric with the distal end portion and the arm portions may be connected to the bearing support portion. Preferably, the bearing support portion may have an axial end facing the first blood flow opening, wherein the axial end of the bearing support portion may preferably be displaced axially inwardly from the first blood flow opening into the direction of the intermediate portion. The bearing support portion is intended to support a bearing of the impeller. Backing the bearing support portion relative to the first blood flow opening further reduces turbulences in the blood flow. In addition, the overlapping distal end portion allows for placing the second sensor as far upstream as possible of the first sensor.

The pump housing may further comprise a first elongated transmitting device, wherein the first sensor may comprise a first axial end and a second axial end. The first elongated transmitting device may be coupled to the second axial end of the first sensor and the first elongated transmitting device may extend to the proximal end portion of the pump housing. Preferably, the first elongated transmitting device is a cable, a fiber, an optical fiber or an optical conductor.

The pump housing may further comprise a first channel extending between the intermediate portion and the proximal end portion of the pump housing. The first elongated transmitting device may be disposed in the first channel. The first channel is preferably recessed from the outer peripheral surface of the intermediate portion and an outer peripheral surface of the proximal end portion of the pump housing. The first channel is preferably lined with a resin, so that the first elongated transmitting device is fixed within the first channel. This greatly reduces the risk of detachment of the first sensor or the first elongated transmitting device when introducing the blood pump into the patient's vascular system. Further, there is no need to guide the first elongated transmitting device within the pump housing and hence, in vicinity to the impeller and the motor.

The second sensor may comprise a first axial end and a second axial end, and the first axial end of the second sensor may be flush with the first blood flow opening in the radial direction. This ensures that the at least one parameter is sensed directly at the first blood flow opening. In particular, this allows for a precise measurement of the suction pressure.

The pump housing may further comprise a second elongated transmitting device, wherein the second elongated transmitting device may be coupled to the second axial end of the second sensor. The second elongated transmitting device may extend to the proximal end portion of the pump housing. Preferably, the second elongated transmitting device is a cable, a fiber, an optical fiber or an optical conductor.

The pump housing may further comprise a second channel extending between the distal end portion and the proximal end portion of the pump housing. The second elongated transmitting device may be disposed in the second channel. The distal end portion of the pump housing may comprise an outer peripheral surface and the proximal end portion of the pump housing may comprise an outer peripheral surface. The second channel may be recessed from the outer peripheral surfaces of the distal end portion, the intermediate portion and the proximal end portion of the pump housing. Preferably, the second channel is lined with a resin, so that the second elongated transmitting device is fixed within the second channel. This greatly reduces the risk of detachment of the second sensor or the second elongated transmitting device when introducing the blood pump into the patient's vascular system. Further, there is no need to guide the second elongated transmitting device within the pump housing and hence, in vicinity to the impeller and the motor.

The proximal end portion of the pump housing may have a smaller diameter than the intermediate portion of the pump housing. The intermediate portion of the pump housing may taper into the proximal end portion of the pump housing. This allows for sufficient space within the pump housing to house e.g. the motor and the impeller. Further, the proximal end portion may comprise a catheter attachment portion, which thus has a smaller diameter than the intermediate portion.

The first sensor may be an optical sensor. The second sensor may be an optical sensor. In particular, the first sensor and/or the second sensor may be fiber-optic sensors, preferably intrinsic fiber-optic sensors. Accordingly, the parameters to be sensed can easily be measured, preferably the aortic pressure and the pressure at the first blood flow opening.

According to a second aspect, a blood pump comprises a pump housing as described above. The blood pump may be a catheter pump or an intravascular blood pump.

### BRIEF DESCRIPTION OF THE DRAWINGS

The foregoing summary, as well as the following detailed description of exemplary embodiments, will be better understood when read in conjunction with the appended drawings. For the purpose of illustrating the present disclosure, reference is made to the drawings. The scope of the disclosure is not limited, however, to the specific embodiments disclosed in the drawings.

In the drawings:

FIG. 1 depicts a side view of a blood pump comprising a pump housing with thereto attached schematically shown cannula and catheter;

FIG. 2 depicts a side view of the pump housing of the blood pump shown in FIG. 1;

FIG. 3 depicts a perspective view of the pump housing shown in FIG. 2;

FIG. 4 depicts a detail of a distal end portion of the pump housing shown in FIG. 2;

FIG. 5 depicts a detail of a first sensor of a pump housing shown in FIG. 2; and

FIG. 6 depicts a cross section of the distal end portion of the pump housing shown in FIG. 2.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, methods, and devices described herein, certain illustrative examples will be described. Although various examples may describe intravascular blood pumps, it will be understood that the improvements of the present technology may also be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices. As is known, intravascular blood pumps can be introduced into a patient, either surgically or percutaneously, to deliver blood from one location in the heart or circulatory system to another location in the heart or circulatory system. For example, when deployed in the left ventricle, an intravascular blood pump can pump blood from the left ventricle of the heart into the aorta. When deployed in the right ventricle, an intravascular blood pump can pump blood from the inferior vena cava into the pulmonary artery.

Herein, “proximal” and “distal” are seen relative to a physician. Thus, proximal designates something which is relatively close to the physician whereas distal designates something which is relatively far away from the physician when the intravascular blood pump is introduced into the patient's body.

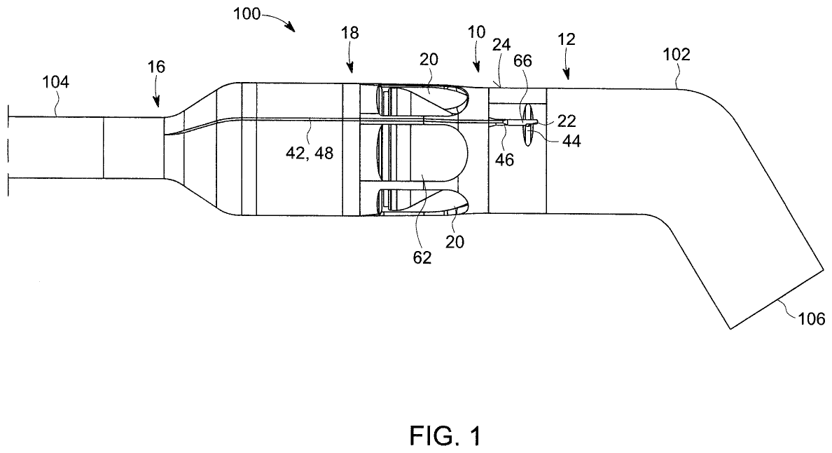

Referring to FIG. 1 a side view of a blood pump 100 is illustrated. The blood pump 100 is designed as an intravascular blood pump and is deployed into the patient's body via a catheter 102 in a known manner. The blood pump 100 comprises a pump housing 10, the catheter 102 and a cannula 104. The catheter 102 is attached to a proximal end portion 16 of the pump housing 10 and the cannula is attached to a distal end portion 12 of the pump housing 10.

The illustrated intravascular blood pump 100 is used as a left ventricle assist device and is introduced percutaneously during a cardiac procedure through the vascular system of a patient. When installed, the cannula 104 reaches through aortic valve into the left ventricle of the heart. The pump housing 10 is located outside of the aortic valve in the aorta. A pump element 62 in form of an impeller is driven by not shown motor and rotates within the pump housing 10 to generate a suction pressure. Thus, blood is unloaded from the left ventricle by entering an inlet 106 of the cannula 104 and exiting the pump housing 10 via a plurality of second blood flow openings 20 in form of blood flow outlets 20, as generally known.

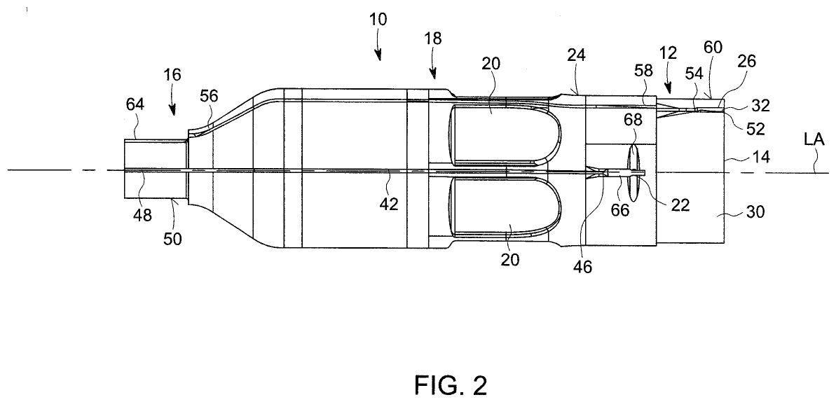

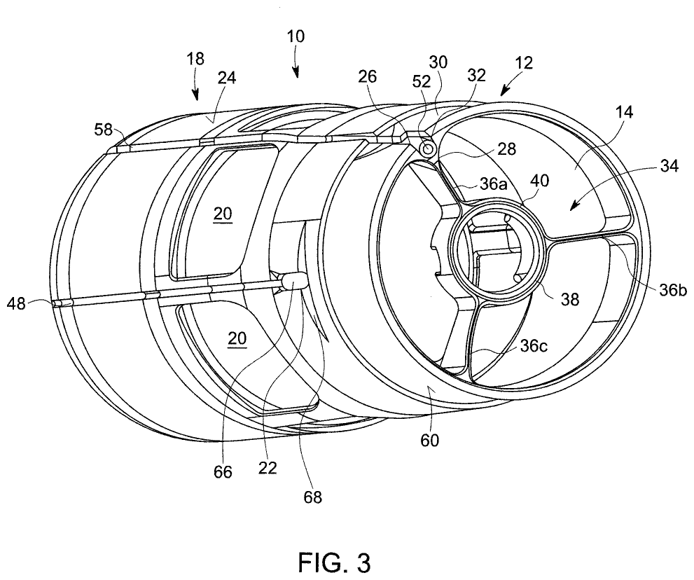

FIGS. 2 and 3 depict the pump housing 10. As shown, an intermediate portion 18 extends between the distal end portion 12 and the proximal end portion 16 and the pump housing 10 has an overall cylindrical shape. The proximal end portion 16 has a smaller diameter than the intermediate portion 18. Thus, the intermediate portion 18 tapers into the proximal end portion 16. The proximal end portion comprises a catheter attachment portion 64 and has an outer peripheral surface 50 configured to support the catheter 104. The pump housing 10 has a central longitudinal axis LA and the terms “radially”, “axially” and the like used herein a relative to the longitudinal axis LA of the pump housing 10.

The distal end portion 12 comprises a cannula attachment portion 30 having an outer peripheral surface 60 configured to support the cannula 102. The cannula attachment portion 30 has a diameter slightly smaller than the intermediate portion 18. As shown in FIG. 1, the outer peripheral surface of the cannula 102 is flush with an outer peripheral surface 24 of the intermediate portion 18, when the cannula 102 is supported on the cannula attachment portion 30.

In the embodiment shown, the intermediate portion 18 comprises six blood flow outlets 20 in total, which are evenly distributed about the circumference of the pump housing 10. The blood enters into the pump housing 10 through a first blood flow opening 14 provided at the axial end of the distal end portion 12. Thus, in this embodiment the first blood flow opening is a blood flow inlet 14. A support member 34 is disposed within the distal end portion 12. In the embodiment shown, the support member 34 comprises a bearing support portion 38 and three arm portions 36a, 36b and 36c. The bearing support portion 38 is concentric with the distal end portion 12 and the longitudinal axis LA. The bearing support portion 38 is configured to support the impeller 62 in a known manner.

The arm portions 36a, 36b and 36c each extend radially inwardly from the cannula attachment portion 30. As shown, the arm portions 36a, 36b and 36c and the bearing support portion 38 are integrally formed with the distal end portion 12. Of course, the entire support member 34 or parts thereof may also be separately formed from the distal end portion 12. Although the embodiment shown comprises three arm portions 36a, 36b and 36c, the support member 34 may also comprise only two arm portions or more than three arm portions.

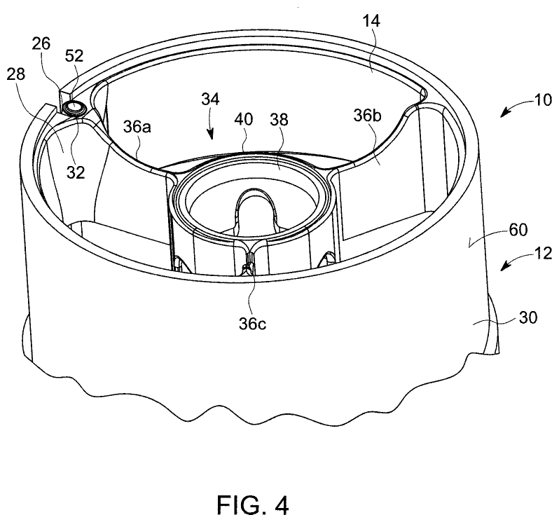

Further, the bearing support portion 38 comprises an axial end 40 facing the blood flow inlet 14. The axial end 40 is not flush with face of the distal end portion 12 or blood flow inlet 14 respectively, but is displaced axially inwardly from the blood flow inlet 14 in direction towards the intermediate portion 18, see also FIG. 4. Backing the bearing support portion 38 reduces turbulences in the blood flow entering the pump housing 10 via the blood flow inlet 14.

As shown in FIG. 4, the arm portions 36a, 36b and 36c enlarge axially in the radial direction, so that an outer radial end of each arm portion 36a, 36b and 36c has an axial extension greater than an inner radial end. Here, the outer radial end of the respective arm portion 36a, 36b and 36c is the end where the arm portions 36a, 36b and 36c are connected to the cannula attachment portion 30 and the inner radial end of the respective arm portion 36a, 36b and 36c is the end where the arm portions 36a, 36b and 36c are connected to the bearing support portion 38. Accordingly, each of the arm portions 36a, 36b and 36c have a sloped shape at the axial end facing the blood flow inlet 14 and a straight shape at the opposite axial end. Further, one of the arm portions 36a comprises a thickened portion 28 at its outer radial end, as will be described below in more detail.

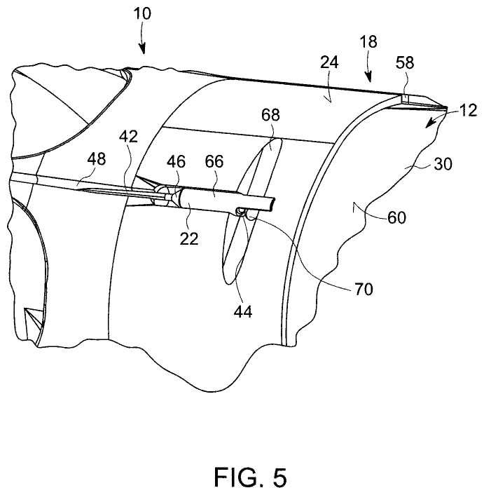

As one can take from FIGS. 1, 2, 3 and 5, the pump housing 10 comprises a first sensor 22 disposed on the outer peripheral surface 24 of the intermediate portion 18. Here, the first sensor 22 is an optical sensor intended to sense at least one parameter, in particular the aortic pressure. As shown, the first sensor 22 is disposed between the blood flow outlets 20 and the distal end portion 12. The first sensor 22 is disposed in a recess and comprises a first axial end 44 and a second axial 46. The first axial end 44 of the first sensor 22 points towards the distal end portion 12 and the second axial end 46 points towards the proximal end portion 16.

The first sensor 22 has a cylindrical shape and is orientated in parallel with the longitudinal axis LA of the pump housing 10. The outer peripheral surface 24 of the intermediate portion 18 further comprises a partially circumferential slot 68 and the first axial end 44 of the first sensor 22 opens into the slot 68. The slot 68 comprises a through hole 70 reaching through the pump housing 10. The first sensor 22 is covered by a shield 66 which protects the first sensor 22 from damage. The slot 68 and the through hole 70 warrant a sufficient blood exchange, so that no blood accumulates in front of the first axial end 44 of the first sensor 22 which might otherwise lead to incorrect parameter sensing.

A first elongated transmitting device 42 in form of an optical fiber is connected to the second axial end 46 of the first sensor 22. The first optical fiber 42 is disposed in a first channel 48 extending from the intermediate portion 18 to the proximal end portion 16. In particular, the first channel 48 is recessed from the outer peripheral surface 24 of the intermediate portion 18 and from the outer peripheral surface 50 of the proximal end portion 16. The first channel 48 is lined with a resin so that the first optical fiber 42 is fixed within the first channel 48. The first optical fiber 42 is thus guided in the first channel 48 up to the end of the proximal end portion 16. There, the first channel 48 opens into the catheter 104 and the first optical fiber 42 is further guided in the catheter 104 in a known manner.

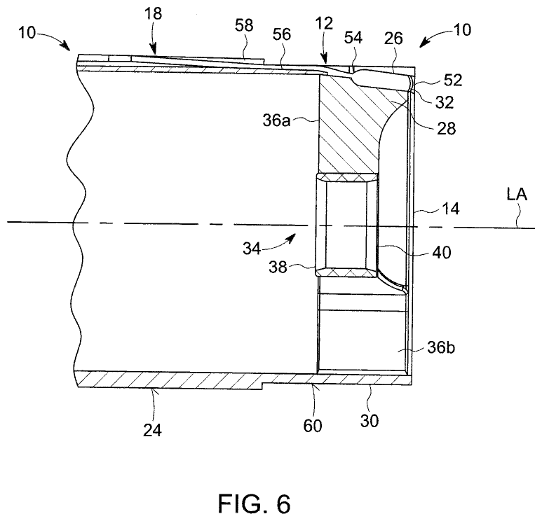

FIG. 6 is a cross section of the pump housing 10 through the arm portion 36a. As one can take from FIG. 6, the pump housing 22 comprises in addition to the first sensor 22 a second sensor 26 for sensing a further parameter, in particular for sensing the pressure at the blood flow inlet 14, i.e. the suction pressure. In this embodiment, the second sensor 26 is identical to the first sensor 22 and is an optical sensor. Of course, the second sensor 26 may also be different from the first sensor 26 if necessary and meaningful.

The second sensor 26 is of cylindrical shape and comprises a first axial end 52 and a second axial end 54. The second sensor 26 is disposed on the distal end portion 12. In particular, the second sensor 26 is disposed in the thickened portion 28 of the arm portion 36a, see FIGS. 4 and 6. Therefore, the thickened portion 28 comprises a support recess 32 supporting the second sensor 26, so that the first axial end 52 of the second sensor 26 is flush with the blood flow inlet 14. Thus, the second sensor 26 is so arranged that it is ensured that the suction pressure is sensed directly at the blood flow inlet 14.

The thickened portion 28 tapers in the axial direction from the blood flow inlet 14 to the intermediate portion 18, see FIG. 4. Thus, the thickened portion 28 has a greater extension in vicinity to the blood flow inlet 14 to securely support the second sensor 26 and a smaller extension in direction towards the intermediate portion 18. In that the thickened portion 28 tapers in the axial direction, turbulences in the blood flow entering the pump housing 10 through the blood flow inlet 14 can be reduced. Further, due to the reduced axial extension of the second sensor 26 a uniform thickness of the thickened portion 28 is not necessary.

The second sensor 26 is disposed in the support recess 32 so that it is inclined relative to the longitudinal axis LA of the pump housing 10. As shown in FIG. 6, the second sensor 26 is inclined in that the second axial end 54 of the second sensor 26 is more remote to the longitudinal axis LA of the pump housing 10 than the first axial end 52 of the second sensor 26.

A second elongated transmitting device 56 in form of an optical fiber is connected to the second axial end 54 of the second sensor 26. A second channel 58 extends from the distal end portion 12 over the intermediate portion 18 to the proximal end portion 16, see also FIG. 2. The second channel 58 is recessed from the outer peripheral surface 60 of the distal end portion 12, the outer peripheral surface 24 of the intermediate portion 18 and the outer peripheral surface 50 of the proximal end portion 16. The second channel 58 is connected to the support recess 23 and the second optical fiber 56 is disposed within the second channel 58. The second channel 68 is lined with a resin so that the second optical fiber 56 is fixed within the second channel 58. Thus, the second optical fiber 56 is securely guided in the second channel 58 from the distal end portion 12 to the proximal end portion 16, where the second channel 58 opens into the catheter 104. The second optical fiber 56 is further guided in the catheter 104 in a conventional and known manner.

As one can take from FIG. 2, the first channel 48 and the second channel 58 both are also recessed from the outer peripheral surface 24 of the intermediate portion 18 tapering into the proximal end portion 16. Due to the first channel 48 and the second channel 58 being lined with resin, detachment of the first optical fiber 42 and the second optical fiber 56 is inhibited.

In contrast to the first sensor 22, the second sensor 26 is not covered by a shield. When the blood pump 100 is assembled, the second sensor 26 is disposed radially inwardly of the cannula 102 and thus, detachment of the second sensor 26 from the pump housing 10 is inhibited.

When the blood pump 100 is correctly installed as a left ventricular assist device, the blood flow through the cannula 104 into the blood flow inlet 14 of the pump housing 10 passes the second sensor 26. Hence, the second sensor 26 can reliably sense the suction pressure. The blood unloaded from the left ventricle exists the blood pump 100 through the blood flow outlets 20 of the pump housing 10 into the aorta and passes the first sensor 22. As such, the first sensor 22 can reliably sense the aortic pressure. In essence, the blood pump 100 allows to directly detect malfunctions and suction events which is vital for the patient's health.

### EXEMPLARY IMPLEMENTATIONS

As already described, the technology described herein may be implemented in various ways. In that regard, the foregoing disclosure is intended to include, but not be limited to, the systems, methods, and combinations and subcombinations thereof that are set forth in the following exemplary implementations. Preferred embodiments are described in the following paragraphs:
A1 Pump housing (10) for a blood pump (100) comprising:
   a distal end portion (12) having a first blood flow opening (14);
   a proximal end portion (16);
   an intermediate portion (18) extending axially between the distal end portion (12) and the proximal end portion (16), the intermediate portion (18) having at least one second blood flow opening (20); and
   a first sensor (22) for sensing at least one parameter, in particular aortic pressure; wherein the first sensor (22) is disposed on an outer peripheral surface (24) of the intermediate portion (18),
   wherein the pump housing (10) comprises a second sensor (26), wherein the second sensor (26) is disposed on the distal end portion (12) for sensing at least one parameter, in particular pressure at the first blood flow opening.
A2 Pump housing (10) according to paragraph A1,
   wherein the distal end portion (12) comprises a thickened portion (28), the thickened portion (28) extending radially inwardly from the distal end portion (12), wherein the second sensor (26) is disposed in the thickened portion (28), wherein the distal end portion (12) preferably comprises a cannula attachment portion (30), the thickened portion (28) extending radially inwardly from the cannula attachment portion (30).
A3 Pump housing (10) according to paragraph A2,
   wherein the thickened portion (28) comprises a support recess (32), wherein the second sensor (26) is disposed in the support recess (32).
A4 Pump housing (10) according to paragraph A2 or A3,
   wherein the thickened portion (28) tapers in the axial direction from the first blood flow opening (14) to the intermediate portion (18).
A5 Pump housing (10) according to any one of the preceding paragraphs A2 to A4,
   wherein a support member (34) with at least two arm portions (36a-36c) is disposed in the distal end portion (12), wherein one of the arm portions (36a) comprises the thickened portion (28).
A6 Pump housing (10) according to paragraph A6,
   wherein the support member (34) comprises a bearing support portion (38) being concentric with the distal end portion (12), the arm portions being (36a-36c) connected to the bearing support portion (38), wherein the bearing support portion (38) preferably has an axial end (40) facing the first blood flow opening (14), wherein the axial end (40) of the bearing support portion (38) is preferably displaced axially inwardly from the first blood flow opening (14) into the direction of the intermediate portion (18).
A7 Pump housing (10) according to any one of the preceding paragraphs A1 to A6,
   wherein the pump housing (10) further comprises a first elongated transmitting device (42), wherein the first sensor comprises (22) a first axial end (44) and a second axial end (46), and wherein the first elongated transmitting (42) device is coupled to the second axial end (46) of the first sensor (22), the first elongated transmitting device (42) extending to the proximal end portion (16) of the pump housing (10), wherein the first elongated transmitting device (42) is preferably a cable, a fiber or an optical conductor.
A8 Pump housing (10) according to paragraph A7,
   wherein the pump housing (10) further comprises a first channel (48) extending between the intermediate portion (18) and the proximal end portion (16) of the pump housing (10), wherein the first elongated transmitting device (42) is disposed in the first channel (48), wherein the first channel (48) is preferably recessed from the outer peripheral surface (24) of the intermediate portion (18) and an outer peripheral surface (50) of the proximal end portion (16) of the pump housing (10), wherein the first channel (48) is preferably lined with a resin, so that the first elongated transmitting device (42) is fixed within the first channel (48).
A9 Pump housing (10) according to any one of the preceding paragraphs A1 to A8,
   wherein the second sensor (26) comprises a first axial end (52) and a second axial end (54), wherein the first axial end (52) of the second sensor (26) is flush with the first blood flow opening (14) in the radial direction.
A10 Pump housing (10) according to paragraph A9,
   wherein the pump housing (10) further comprising a second elongated transmitting device (56), wherein the second elongated transmitting device (56) is coupled to the second axial end (54) of the second sensor (26), the second elongated transmitting device (56) extending to the proximal end portion (14) of the pump housing (10), wherein the second elongated transmitting device (56) is preferably a cable, a fiber or an optical conductor.
A11 Pump housing (10) according to paragraph A10,
   wherein the pump housing (10) further comprises a second channel (58) extending between the distal end portion (12) and the proximal end portion (14) of the pump housing (10), wherein the second elongated transmitting device (58) is disposed in the second channel (58).
A12 Pump housing (10) according to paragraph A11,
   wherein the distal end portion (12) of the pump housing comprises an outer peripheral surface (60) and the proximal end portion (16) of the pump housing (10) comprises an outer peripheral surface (50), wherein the second channel (58) is recessed from the outer peripheral surfaces (24, 50, 60) of the distal end portion (12), the intermediate portion (18) and the proximal end portion (16) of the pump housing (10), wherein the second channel (58) is preferably lined with a resin, so that the second elongated transmitting (56) device is fixed within the second channel (58).
A13 Pump housing (10) according to any one of the preceding paragraphs A1 to A12,
   wherein the proximal end portion (14) of the pump housing (10) has a smaller diameter than the intermediate portion (18) of the pump housing (10), wherein the intermediate portion (18) of the pump housing (10) tapers into the proximal end portion (14) of the pump housing (10).
A14 Pump housing (10) according to any one of the preceding claims A1 to A13,
   wherein the first sensor (22) is an optical sensor and/or wherein the second sensor (26) is an optical sensor.
A15 Pump housing (10) according to any one the preceding paragraphs A1 to A14,
   wherein the pump housing (10) has a longitudinal axis (LA) and the second sensor (22) is inclined relative to the longitudinal axis (LA) of the pump housing (10).
A16 Pump housing (10) according to paragraph A15,
   wherein the second sensor (22) is increasingly inclined relative to the longitudinal axis (LA) of the pump housing (10) from the distal end portion (12) to the intermediate portion (18).
A17 Pump housing (10) according to any one the preceding paragraphs A1 to A16,
   wherein the pump housing (10) comprises a shield (66), wherein the shield (66) is disposed radially outwardly on the first sensor (22), so that the shield (66) shields the first sensor (22).
A18 Pump housing (10) according to any one the preceding paragraphs A1 to A17,
   wherein the intermediate portion (18) of the pump housing (10) comprises a partially circumferential slot (68) and the first sensor (22) is disposed in vicinity to the slot (68) or the first sensor (22) is at least partially disposed within the slot (68).
A19 Pump housing (10) according to paragraph A18, wherein a through hole (70) passes through the slot (68) inside the pump housing (10).
A20 Blood pump (100) comprising a pump housing (10) according to any one of the preceding paragraphs A1 to A19, wherein the blood pump is preferably a catheter pump or an intravascular blood pump.
A21 Blood pump (100) according to paragraph A20,
   wherein the blood pump (100) comprises a cannula (102), wherein the cannula (102) is partially disposed on the distal end portion (12), and wherein the second sensor (26) is disposed radially inwardly of the cannula (102).
A22 Blood pump (100) according to paragraph A21 or A22,
   wherein the blood pump (100) comprises a catheter (104), wherein the catheter (104) is partially disposed on the proximal end portion (14), and wherein the first channel (42) and/or the second channel (58) are partially disposed radially inwardly of the catheter (104).

### LIST OF REFERENCE SIGNS

10 pump housing
12 distal end portion
14 first blood flow opening/blood flow inlet
16 proximal end portion
18 intermediate portion
20 second blood flow opening/blood flow outlet
22 first sensor
24 outer peripheral surface of intermediate portion
26 second sensor
28 thickened portion
30 cannula attachment portion
32 support recess
34 support member
36a arm portion
36 arm portion
36c arm portion
38 bearing support portion
40 axial end of bearing support portion
42 first elongated transmitting device/optical fiber
44 first axial end of first sensor
46 second axial end of first sensor
48 first channel
50 outer peripheral surface of proximal end portion
52 first axial end of second sensor
54 second axial end of second sensor
56 second elongated transmitting device/optical fiber
58 second channel
60 outer peripheral surface of distal end portion
62 pump element/impeller
64 catheter attachment portion
66 shield
68 slot
70 through hole
100 blood pump
102 catheter
104 cannula
106 inlet
LA longitudinal axis of pump housing

1. Pump housing for a blood pump comprising:
a distal end portion having a first blood flow opening;
a proximal end portion;
an intermediate portion extending axially between the distal end portion and the proximal end portion, the intermediate portion having at least one second blood flow opening; and
a first sensor for sensing at least one parameter, in particular aortic pressure;
wherein the first sensor is disposed on an outer peripheral surface of the intermediate portion,
wherein the pump housing comprises a second sensor, wherein the second sensor is disposed on the distal end portion for sensing at least one parameter, in particular pressure at the first blood flow opening.

2. Pump housing according to claim 1,
wherein the distal end portion comprises a thickened portion, the thickened portion, extending radially inwardly from the distal end portion, wherein the second sensor is disposed in the thickened portion, wherein the distal end portion preferably comprises a cannula attachment portion, the thickened portion extending radially inwardly from the cannula attachment portion.

3. Pump housing according to claim 2,
wherein the thickened portion comprises a support recess, wherein the second sensor is disposed in the support recess.

4. Pump housing according to claim 2,
wherein the thickened portion tapers in the axial direction from the first blood flow opening to the intermediate portion.

5. Pump housing according to claim 2,
wherein a support member with at least two arm portions is disposed in the distal end portion, wherein one of the arm portions comprises the thickened portion.

6. Pump housing according to claim 5,
wherein the support member comprises a bearing support portion being concentric with the distal end portion, the arm portions being connected to the bearing support portion, wherein the bearing support portion preferably has an axial end facing the first blood flow opening, wherein the axial end of the bearing support portion is preferably displaced axially inwardly from the first blood flow opening into a direction of the intermediate portion.

7. Pump housing according to claim 1,
wherein the pump housing further comprises a first elongated transmitting device, wherein the first sensor comprises a first axial end and a second axial end, and wherein the first elongated transmitting device is coupled to the second axial end of the first sensor, the first elongated transmitting device extending to the proximal end portion of the pump housing, wherein the first elongated transmitting device is preferably a cable, a fiber or an optical conductor.

8. Pump housing according to claim 7,
wherein the pump housing further comprises a first channel extending between the intermediate portion and the proximal end portion of the pump housing, wherein the first elongated transmitting device is disposed in the first channel, wherein the first channel is preferably recessed from the outer peripheral surface of the intermediate portion and an outer peripheral surface of the proximal end portion of the pump housing, wherein the first channel is preferably lined with a resin, so that the first elongated transmitting device is fixed within the first channel.

9. Pump housing according to claim 1,
wherein the second sensor comprises a first axial end and a second axial end, wherein the first axial end of the second sensor is flush with the first blood flow opening in a radial direction.

10. Pump housing according to claim 9,
wherein the pump housing further comprising a second elongated transmitting device, wherein the second elongated transmitting device is coupled to the second axial end of the second sensor, the second elongated transmitting device extending to the proximal end portion of the pump housing, wherein the second elongated transmitting device is preferably a cable, a fiber or an optical conductor.

11. Pump housing, according to claim 10,
wherein the pump housing further comprises a second channel extending between the distal end portion and the proximal end portion of the pump housing, wherein the second elongated transmitting device is disposed in the second channel.

12. Pump housing according to claim 11,
wherein the distal end portion of the pump housing comprises an outer peripheral surface and the proximal end portion of the pump housing comprises an outer peripheral surface, wherein the second channel is recessed from the outer peripheral surfaces of the distal end portion, the intermediate portion and the proximal end portion of the pump housing, wherein the second channel is preferably lined with a resin, so that the second elongated transmitting device is fixed within the second channel.

13. Pump housing according to claim 1,
wherein the proximal end portion of the pump housing has a smaller diameter than the intermediate portion of the pump housing, wherein the intermediate portion of the pump housing tapers into the proximal end portion of the pump housing.

14. Pump housing according to claim 1,
wherein the first sensor is an optical sensor and/or wherein the second sensor is an optical sensor.

15. Blood pump comprising a pump housing comprising:
a distal end portion having a first blood flow opening;
a proximal end portion;
an intermediate portion extending axially between the distal end portion and the proximal end portion, the intermediate portion having at least one second blood flow opening; and
a first sensor for sensing at least one parameter, in particular aortic pressure;
wherein the first sensor is disposed on an outer peripheral surface of the intermediate portion,
wherein the pump housing comprises a second sensor, wherein the second sensor is disposed on the distal end portion for sensing at least one parameter, in particular pressure at the first blood flow opening, wherein the blood pump is preferably a catheter pump or an intravascular blood pump.
